SEQUENCE LISTING (8 sequences)

SEQ ID NO: 1 - DNA, 5930 bp, Zea mays
  source 1..5930: mol_type = genomic DNA
gatattgaatccaattcaattctataacctccaaaatcgatatcctaacatagcatgatagtttttttgagaggtgtgatttaacatattagatgaattttctttttttaatacgctatgttctagatgtctgacgatgaatggagccgctctgcatataccaaacgctatctaagtttgtgaacaaatgactaaattatccacacgaccacaattgggcgctgataagatccacacttgctggttttaattcatccggtctttatcaactgtcacatcagttatagatcattatcaacttttatctacaattgatgttgtaatacacggtcacttaagaccatgtttaggtacaatgtctctcaaaactatgattttacttatgatgacaataccgtagttttgaatagctctaaaaatatcatggttctaaaaatattgtttggattcaacattataaatcgtggtattaagcaaaagctagtcatgttataaaaactttaggttgaagtagagttttcaatactaaaaaaatatcatggtatttagaatatcatgattttaaaaatatagttcttacgaatgcaaccaaacaccttatgatataaaataatatggtattgcctacaaactacaaaaataaatcactcccaagcattacataaacatcatttttataaactttttagtaaaataaagcgcaatgtttttattaaatagaaatttatacaagtatatataattaagcaaataaaaactacgttattcataaaaagctaataaaaagtaaaaagtaaaaactatataattcataaaaaaatcgttatctttctcttccatcttattatctatttttagttgttttgtcaaatttaaggctattttatgggttctttttatctttgtacatgtcgtgagccaataacttattgaattggtcaatcaatttaggttgagttttacttttaactaacctagtatagtttgttcaacacctttttttcataatcggggactcattccacttgttgttgacccattttttcttctactgagttcttgcccttttgcaatcaataggacgtggtgagcccatatttttaggtgttatggatttataatcaccgttcaaatttgaatgaatattttgtttttattattgaagtcttcgtcaatcgttatcatgcctcgcataccacttagaccgacgaagtccaacatatttctcccccgacgaggagagattcttcgtaaaattgctactggtattactggtattgaatagaagataagggtgcgtttggttgcatgcatctacgcgtgcttgcatcacggaatgcgggttggtgctgtttggttgctataagctagactatacatatgcaagttttgtgtttggttggctgcatgcagatttcgatccaaactcgcacgatccacacagcaccctgggccaggcttaccagatacgagtagattggtcgcatctttggagccaggctttagcggtccgcggtcctggctgacacggccagattgccgagagaaaccaaccaaacagggtctaaatgtccaatcgtttgactgctactggatgaaaaaagaatgtcacaactttaaaatgtgtatttatttatactcctacacatgaaataactactatactcagatttcttttacattcagattttttctcagccactgaaacataccagccctttactaccaaaaacaggaactccacggtccaatgattatgtgagtcggaggagaggggaggaagaatcgcctgtgaaggagggagggagggagggacgtccgatccgaagatggaaggagctggggaggggagcgccttgacggggatgatgggtcctgcgctcgacaagctcgctagcctcgttgacaagtacaccgagctcagaaacgtgaggaagaagatggagcagctgaggaaggagctgattgcgatcaacctcgcgcttgagaagcacgcggccatggagaacccagacgcgcaggcgaaggcgtgggcggcggagatgcgcgagctggcctacgacatggaggacagcatcgatctcttcacccaccacgtcgaccacgaaccggccgacaccgccaccaccggcgtcaagaggttcttcctccggatcatccggaagcttaagaaactccactaccgccacaggtttgttcaggagatcaaacaactccacgaccttgccaacgaatcgtaccggcgtaggaagaggtacaggattgaggagggcggttcaagcctctcgcacgcggagatcgatcctcggttagaggcgctctacgtggaggtggagaaactcgtgggcatccagggcccaagccaggagatcattggacagctcgtcggcgagaacgcagcggagcgacggagggttgtcgccgttgttggatctggaggttcaggcaagaccacacttgccaaacaggtgtacgagaaaatcaggtgccaattctcttgtgcagcctttgtgtctgtgtcgcaaaagcccaacatgaatagcctcctgtgggagttgctatctcaaatcgggaaccatggtggagatttaggaatgatggcagtaggatattgcagtgacaaacaactgatcgacagactaagatcacatcttgaaaagcagaggttagtttaccttttcattccggttagcttaattcggtacaccaactagagatttgtgatttgctattaattacaccaaatttctcctacacaacaataactggtttagcatgatggcgatccaaagtcaaaactatcttctactactagtgtatgccatactcatatagatattttcttttcataaactctcgtagcatttttacatgcattcatattcctattgcctttatacagaactgatttttcactgcttcacaatctgctcttaggtatctcgttgtgatagatgatgtttggacaaactcagcgtgggagaccatacaatgtgcgctccctaaaaatgcccatgcaagtaaaataattctgacaacacgaatcaacagtgtaggccagttctcctgcactccagatgagggttttatctatcagatgaagcctctttgcagaaacgattctgaaaatctgtttctgaaaaggacactatgtgataaagataagtttcctgctcagctggaggggattaaaaacgagataatcgagaaatgcgatggtttgccactggctattgttactctagctagcatgttagctactaaacagagaacaagggaagaatgggagagggcacttgattcaatccattctatgcacaagaaagatagtggcctggaagtgatggacaagatactgtctctgagttacagggatctacctcacaacatgagaaattgcttgctgtatctcagtacatttccagaggaccacacgatttacaaagatgccctagtatggagatggatggctgaagggtttatcgctgaaacacaaggctttactttggagcaggttgccgagggctacttctacgagtttgtgaacaggagtttggttcagcccataaccttgcgttcaagatatgaaatgcgtggagaaggaggttgccgagtccatgacattgtactgaacttcctcatctctcgtgcagctgaagagaactttttaactacgctgtatggcgcccagggggttccatcttcagaccgaaggattcgccggctctctgtctgggacagtccagaacacgcactggcagtctctagagcgaccatgaatctgtcccatctccggtcagttagaatatgcaacgttggagactggcccgtgcctgctgttctagacttacctgtccttcgagtgttagatctagagggatgccgtgatctgaggatcgacgaacctgactgcattctaagcttgtttcatctgagatacctgggtttccgcagcgcaagtggtgtcgtgctaccggctcaaatcggaaatttacaccatctgcagaccatcgatttaagcgggactggagtgacacagctgccagaaagcattgtccagctcaagcgactgatgcatcttgttgggcaacggctcatcatgccagacgggtttggtagcatggaatcccttgaggagttaggtactatcgactgctgcaagtgccccgtcagttttggggaagacctagcacttctgagcaggctgagggtgctccgagtggctttcatcggggtcgaaacaagtgacatggaaaccagaaggaaatctttgatgtcatccctctgcaaactcggaggagacaaccttcggcgtgtcactattatcgacctcgctggcggtggagattgctttgtggagtcgtggcaccctcctcctcgtctcctccagaagttcatccatatcagtcagcaacagcacttctccaggtttccagaatggatcagttcctgcctatgtgatctcacccacctggatataaaggccgaaaagatggaaagggagcatctaagtgttcttgaacacctgcccgccatccgttgcctataccttttcgtgaagcgagtctccgaagacgggctcgccatcagccacggcgcgttccgatgtctacggcgtctcgagttctgcaacgtagatggacctggtttgatgtttgcaggaggcgttccaatgttggaatggctgaggctcgggttcgacgcggatagagcgcaatcgacatacggcggtctggaggttggcatccagcgcctctcgtctctcaaacatgtcgtgctcattgtatggatggtttctgaaggcggtgatgatccagcggagcaagccgtctggtctgccatcaatggccaagtagagatgctccccaactctccgacggttgatatccggtttcgtagacggagtcagctgcaggcaagctcagaataaggagcacgaaaaagacgatgatgttggatgtcgcctgctagctgtagtatgttgctgcttctgcttgttgccaacacattttttttgggttagggtggggtacaaccataaaatgtgtgtggatgtgcttgtaagcattacttgtatgtttttttttgtaaagcacaatatagatagatgcatatatgtgtgcgtgcaaagctatgattatcgacactcacacttgtacattagctagatgaaggtctcgacagagcagagcatagtacacatctctgggagttggactggacactataacggggatgctgcagccgaaactcaaaagctacatgcatgtcacttggctcatcggcgcaggaagcaacgcaggtccatcacgcgcagcaccttgaacccgtccaggctacaccccagcagcagcctcggcttcttcggcgccggcgggcagcgcggggggcgttcggttccagcgccactcgccgtcgccgacgacagcggcgagagcttccgtggaccttcttcgtcctcgtcgccgccgcgggagtaagaccgcttcagcttcagcggccgcagctccaccagctgcggaatgctaacatcttcttcaggcacctgccgggtggttctctttcccagaatcttccgctggagacccatggcgagctcggttcggtttaacctgcaattaatgcgcaatagcggagtaggaagcgagattgttagctcaggcgagcagagatgaaccggctaggaaagtttagttgctcgtgcatgctaccagctagcttgttggaggctcttgctttggacaccaagcaacggatatgacagcagaatgtgcgtgcttataagcaagcaagcagagcagcagttgcaaagaagggaaaaggtggagtggaaaaaggagttgccattgaagtgcacgagtgagcgagcatcatgtcaaggaaccaggagggaagttgcacaacagatgaaaagcggagagctgtttccg

SEQ ID NO: 2 - DNA, 2829 bp, Zea mays
  source 1..2829: mol_type = other DNA
atggaaggagctggggaggggagcgccttgacggggatgatgggtcctgcgctcgacaagctcgctagcctcgttgacaagtacaccgagctcagaaacgtgaggaagaagatggagcagctgaggaaggagctgattgcgatcaacctcgcgcttgagaagcacgcggccatggagaacccagacgcgcaggcgaaggcgtgggcggcggagatgcgcgagctggcctacgacatggaggacagcatcgatctcttcacccaccacgtcgaccacgaaccggccgacaccgccaccaccggcgtcaagaggttcttcctccggatcatccggaagcttaagaaactccactaccgccacaggtttgttcaggagatcaaacaactccacgaccttgccaacgaatcgtaccggcgtaggaagaggtacaggattgaggagggcggttcaagcctctcgcacgcggagatcgatcctcggttagaggcgctctacgtggaggtggagaaactcgtgggcatccagggcccaagccaggagatcattggacagctcgtcggcgagaacgcagcggagcgacggagggttgtcgccgttgttggatctggaggttcaggcaagaccacacttgccaaacaggtgtacgagaaaatcaggtgccaattctcttgtgcagcctttgtgtctgtgtcgcaaaagcccaacatgaatagcctcctgtgggagttgctatctcaaatcgggaaccatggtggagatttaggaatgatggcagtaggatattgcagtgacaaacaactgatcgacagactaagatcacatcttgaaaagcagaggtatctcgttgtgatagatgatgtttggacaaactcagcgtgggagaccatacaatgtgcgctccctaaaaatgcccatgcaagtaaaataattctgacaacacgaatcaacagtgtaggccagttctcctgcactccagatgagggttttatctatcagatgaagcctctttgcagaaacgattctgaaaatctgtttctgaaaaggacactatgtgataaagataagtttcctgctcagctggaggggattaaaaacgagataatcgagaaatgcgatggtttgccactggctattgttactctagctagcatgttagctactaaacagagaacaagggaagaatgggagagggcacttgattcaatccattctatgcacaagaaagatagtggcctggaagtgatggacaagatactgtctctgagttacagggatctacctcacaacatgagaaattgcttgctgtatctcagtacatttccagaggaccacacgatttacaaagatgccctagtatggagatggatggctgaagggtttatcgctgaaacacaaggctttactttggagcaggttgccgagggctacttctacgagtttgtgaacaggagtttggttcagcccataaccttgcgttcaagatatgaaatgcgtggagaaggaggttgccgagtccatgacattgtactgaacttcctcatctctcgtgcagctgaagagaactttttaactacgctgtatggcgcccagggggttccatcttcagaccgaaggattcgccggctctctgtctgggacagtccagaacacgcactggcagtctctagagcgaccatgaatctgtcccatctccggtcagttagaatatgcaacgttggagactggcccgtgcctgctgttctagacttacctgtccttcgagtgttagatctagagggatgccgtgatctgaggatcgacgaacctgactgcattctaagcttgtttcatctgagatacctgggtttccgcagcgcaagtggtgtcgtgctaccggctcaaatcggaaatttacaccatctgcagaccatcgatttaagcgggactggagtgacacagctgccagaaagcattgtccagctcaagcgactgatgcatcttgttgggcaacggctcatcatgccagacgggtttggtagcatggaatcccttgaggagttaggtactatcgactgctgcaagtgccccgtcagttttggggaagacctagcacttctgagcaggctgagggtgctccgagtggctttcatcggggtcgaaacaagtgacatggaaaccagaaggaaatctttgatgtcatccctctgcaaactcggaggagacaaccttcggcgtgtcactattatcgacctcgctggcggtggagattgctttgtggagtcgtggcaccctcctcctcgtctcctccagaagttcatccatatcagtcagcaacagcacttctccaggtttccagaatggatcagttcctgcctatgtgatctcacccacctggatataaaggccgaaaagatggaaagggagcatctaagtgttcttgaacacctgcccgccatccgttgcctataccttttcgtgaagcgagtctccgaagacgggctcgccatcagccacggcgcgttccgatgtctacggcgtctcgagttctgcaacgtagatggacctggtttgatgtttgcaggaggcgttccaatgttggaatggctgaggctcgggttcgacgcggatagagcgcaatcgacatacggcggtctggaggttggcatccagcgcctctcgtctctcaaacatgtcgtgctcattgtatggatggtttctgaaggcggtgatgatccagcggagcaagccgtctggtctgccatcaatggccaagtagagatgctccccaactctccgacggttgatatccggtttcgtagacggagtcagctgcaggcaagctcagaataa

SEQ ID NO: 3 - protein, 942 aa, Zea mays
MEGAGEGSALTGMMGPALDKLASLVDKYTELRNVRKKMEQLRKELIAINLALEKHAAMENPDAQAKAWAAEMRELAYDMEDSIDLFTHHVDHEPADTATTGVKRFFLRIIRKLKKLHYRHRFVQEIKQLHDLANESYRRRKRYRIEEGGSSLSHAEIDPRLEALYVEVEKLVGIQGPSQEIIGQLVGENAAERRRVVAVVGSGGSGKTTLAKQVYEKIRCQFSCAAFVSVSQKPNMNSLLWELLSQIGNHGGDLGMMAVGYCSDKQLIDRLRSHLEKQRYLVVIDDVWTNSAWETIQCALPKNAHASKIILTTRINSVGQFSCTPDEGFIYQMKPLCRNDSENLFLKRTLCDKDKFPAQLEGIKNEIIEKCDGLPLAIVTLASMLATKQRTREEWERALDSIHSMHKKDSGLEVMDKILSLSYRDLPHNMRNCLLYLSTFPEDHTIYKDALVWRWMAEGFIAETQGFTLEQVAEGYFYEFVNRSLVQPITLRSRYEMRGEGGCRVHDIVLNFLISRAAEENFLTTLYGAQGVPSSDRRIRRLSVWDSPEHALAVSRATMNLSHLRSVRICNVGDWPVPAVLDLPVLRVLDLEGCRDLRIDEPDCILSLFHLRYLGFRSASGVVLPAQIGNLHHLQTIDLSGTGVTQLPESIVQLKRLMHLVGQRLIMPDGFGSMESLEELGTIDCCKCPVSFGEDLALLSRLRVLRVAFIGVETSDMETRRKSLMSSLCKLGGDNLRRVTIIDLAGGGDCFVESWHPPPRLLQKFIHISQQQHFSRFPEWISSCLCDLTHLDIKAEKMEREHLSVLEHLPAIRCLYLFVKRVSEDGLAISHGAFRCLRRLEFCNVDGPGLMFAGGVPMLEWLRLGFDADRAQSTYGGLEVGIQRLSSLKHVVLIVWMVSEGGDDPAEQAVWSAINGQVEMLPNSPTVDIRFRRRSQLQASSE

SEQ ID NO: 4 - DNA, 2658 bp, Zea mays
  source 1..2658: mol_type = other DNA
atggagaacccagacgcgcaggcgaaggcgtgggcggcggagatgcgcgagctggcctacgacatggaggacagcatcgatctcttcacccaccacgtcgaccacgaaccggccgacaccgccaccaccggcgtcaagaggttcttcctccggatcatccggaagcttaagaaactccactaccgccacaggtttgttcaggagatcaaacaactccacgaccttgccaacgaatcgtaccggcgtaggaagaggtacaggattgaggagggcggttcaagcctctcgcacgcggagatcgatcctcggttagaggcgctctacgtggaggtggagaaactcgtgggcatccagggcccaagccaggagatcattggacagctcgtcggcgagaacgcagcggagcgacggagggttgtcgccgttgttggatctggaggttcaggcaagaccacacttgccaaacaggtgtacgagaaaatcaggtgccaattctcttgtgcagcctttgtgtctgtgtcgcaaaagcccaacatgaatagcctcctgtgggagttgctatctcaaatcgggaaccatggtggagatttaggaatgatggcagtaggatattgcagtgacaaacaactgatcgacagactaagatcacatcttgaaaagcagaggtatctcgttgtgatagatgatgtttggacaaactcagcgtgggagaccatacaatgtgcgctccctaaaaatgcccatgcaagtaaaataattctgacaacacgaatcaacagtgtaggccagttctcctgcactccagatgagggttttatctatcagatgaagcctctttgcagaaacgattctgaaaatctgtttctgaaaaggacactatgtgataaagataagtttcctgctcagctggaggggattaaaaacgagataatcgagaaatgcgatggtttgccactggctattgttactctagctagcatgttagctactaaacagagaacaagggaagaatgggagagggcacttgattcaatccattctatgcacaagaaagatagtggcctggaagtgatggacaagatactgtctctgagttacagggatctacctcacaacatgagaaattgcttgctgtatctcagtacatttccagaggaccacacgatttacaaagatgccctagtatggagatggatggctgaagggtttatcgctgaaacacaaggctttactttggagcaggttgccgagggctacttctacgagtttgtgaacaggagtttggttcagcccataaccttgcgttcaagatatgaaatgcgtggagaaggaggttgccgagtccatgacattgtactgaacttcctcatctctcgtgcagctgaagagaactttttaactacgctgtatggcgcccagggggttccatcttcagaccgaaggattcgccggctctctgtctgggacagtccagaacacgcactggcagtctctagagcgaccatgaatctgtcccatctccggtcagttagaatatgcaacgttggagactggcccgtgcctgctgttctagacttacctgtccttcgagtgttagatctagagggatgccgtgatctgaggatcgacgaacctgactgcattctaagcttgtttcatctgagatacctgggtttccgcagcgcaagtggtgtcgtgctaccggctcaaatcggaaatttacaccatctgcagaccatcgatttaagcgggactggagtgacacagctgccagaaagcattgtccagctcaagcgactgatgcatcttgttgggcaacggctcatcatgccagacgggtttggtagcatggaatcccttgaggagttaggtactatcgactgctgcaagtgccccgtcagttttggggaagacctagcacttctgagcaggctgagggtgctccgagtggctttcatcggggtcgaaacaagtgacatggaaaccagaaggaaatctttgatgtcatccctctgcaaactcggaggagacaaccttcggcgtgtcactattatcgacctcgctggcggtggagattgctttgtggagtcgtggcaccctcctcctcgtctcctccagaagttcatccatatcagtcagcaacagcacttctccaggtttccagaatggatcagttcctgcctatgtgatctcacccacctggatataaaggccgaaaagatggaaagggagcatctaagtgttcttgaacacctgcccgccatccgttgcctataccttttcgtgaagcgagtctccgaagacgggctcgccatcagccacggcgcgttccgatgtctacggcgtctcgagttctgcaacgtagatggacctggtttgatgtttgcaggaggcgttccaatgttggaatggctgaggctcgggttcgacgcggatagagcgcaatcgacatacggcggtctggaggttggcatccagcgcctctcgtctctcaaacatgtcgtgctcattgtatggatggtttctgaaggcggtgatgatccagcggagcaagccgtctggtctgccatcaatggccaagtagagatgctccccaactctccgacggttgatatccggtttcgtagacggagtcagctgcaggcaagctcagaataa

SEQ ID NO: 5 - protein, 885 aa, Zea mays
MENPDAQAKAWAAEMRELAYDMEDSIDLFTHHVDHEPADTATTGVKRFFLRIIRKLKKLHYRHRFVQEIKQLHDLANESYRRRKRYRIEEGGSSLSHAEIDPRLEALYVEVEKLVGIQGPSQEIIGQLVGENAAERRRVVAVVGSGGSGKTTLAKQVYEKIRCQFSCAAFVSVSQKPNMNSLLWELLSQIGNHGGDLGMMAVGYCSDKQLIDRLRSHLEKQRYLVVIDDVWTNSAWETIQCALPKNAHASKIILTTRINSVGQFSCTPDEGFIYQMKPLCRNDSENLFLKRTLCDKDKFPAQLEGIKNEIIEKCDGLPLAIVTLASMLATKQRTREEWERALDSIHSMHKKDSGLEVMDKILSLSYRDLPHNMRNCLLYLSTFPEDHTIYKDALVWRWMAEGFIAETQGFTLEQVAEGYFYEFVNRSLVQPITLRSRYEMRGEGGCRVHDIVLNFLISRAAEENFLTTLYGAQGVPSSDRRIRRLSVWDSPEHALAVSRATMNLSHLRSVRICNVGDWPVPAVLDLPVLRVLDLEGCRDLRIDEPDCILSLFHLRYLGFRSASGVVLPAQIGNLHHLQTIDLSGTGVTQLPESIVQLKRLMHLVGQRLIMPDGFGSMESLEELGTIDCCKCPVSFGEDLALLSRLRVLRVAFIGVETSDMETRRKSLMSSLCKLGGDNLRRVTIIDLAGGGDCFVESWHPPPRLLQKFIHISQQQHFSRFPEWISSCLCDLTHLDIKAEKMEREHLSVLEHLPAIRCLYLFVKRVSEDGLAISHGAFRCLRRLEFCNVDGPGLMFAGGVPMLEWLRLGFDADRAQSTYGGLEVGIQRLSSLKHVVLIVWMVSEGGDDPAEQAVWSAINGQVEMLPNSPTVDIRFRRRSQLQASSE

SEQ ID NO: 6 - protein, 885 aa, Zea mays; identical to SEQ ID NO: 5

SEQ ID NO: 7 - DNA, 3101 bp, Zea mays
  source 1..3101: mol_type = genomic DNA
atggaaggagctggggaggggagcgccttgacggggatgatgggtcctgcgctcgacaagctcgctagcctcgttgacaagtacaccgagctcagaaacgtgaggaagaagatggagcagctgaggaaggagctgattgcgatcaacctcgcgcttgagaagcacgcggccatggagaacccagacgcgcaggcgaaggcgtgggcggcggagatgcgcgagctggcctacgacatggaggacagcatcgatctcttcacccaccacgtcgaccacgaaccggccgacaccgccaccaccggcgtcaagaggttcttcctccggatcatccggaagcttaagaaactccactaccgccacaggtttgttcaggagatcaaacaactccacgaccttgccaacgaatcgtaccggcgtaggaagaggtacaggattgaggagggcggttcaagcctctcgcacgcggagatcgatcctcggttagaggcgctctacgtggaggtggagaaactcgtgggcatccagggcccaagccaggagatcattggacagctcgtcggcgagaacgcagcggagcgacggagggttgtcgccgttgttggatctggaggttcaggcaagaccacacttgccaaacaggtgtacgagaaaatcaggtgccaattctcttgtgcagcctttgtgtctgtgtcgcaaaagcccaacatgaatagcctcctgtgggagttgctatctcaaatcgggaaccatggtggagatttaggaatgatggcagtaggatattgcagtgacaaacaactgatcgacagactaagatcacatcttgaaaagcagaggttagtttaccttttcattccggttagcttaattcggtacaccaactagagatttgtgatttgctattaattacaccaaatttctcctacacaacaataactggtttagcatgatggcgatccaaagtcaaaactatcttctactactagtgtatgccatactcatatagatattttcttttcataaactctcgtagcatttttacatgcattcatattcctattgcctttatacagaactgatttttcactgcttcacaatctgctcttaggtatctcgttgtgatagatgatgtttggacaaactcagcgtgggagaccatacaatgtgcgctccctaaaaatgcccatgcaagtaaaataattctgacaacacgaatcaacagtgtaggccagttctcctgcactccagatgagggttttatctatcagatgaagcctctttgcagaaacgattctgaaaatctgtttctgaaaaggacactatgtgataaagataagtttcctgctcagctggaggggattaaaaacgagataatcgagaaatgcgatggtttgccactggctattgttactctagctagcatgttagctactaaacagagaacaagggaagaatgggagagggcacttgattcaatccattctatgcacaagaaagatagtggcctggaagtgatggacaagatactgtctctgagttacagggatctacctcacaacatgagaaattgcttgctgtatctcagtacatttccagaggaccacacgatttacaaagatgccctagtatggagatggatggctgaagggtttatcgctgaaacacaaggctttactttggagcaggttgccgagggctacttctacgagtttgtgaacaggagtttggttcagcccataaccttgcgttcaagatatgaaatgcgtggagaaggaggttgccgagtccatgacattgtactgaacttcctcatctctcgtgcagctgaagagaactttttaactacgctgtatggcgcccagggggttccatcttcagaccgaaggattcgccggctctctgtctgggacagtccagaacacgcactggcagtctctagagcgaccatgaatctgtcccatctccggtcagttagaatatgcaacgttggagactggcccgtgcctgctgttctagacttacctgtccttcgagtgttagatctagagggatgccgtgatctgaggatcgacgaacctgactgcattctaagcttgtttcatctgagatacctgggtttccgcagcgcaagtggtgtcgtgctaccggctcaaatcggaaatttacaccatctgcagaccatcgatttaagcgggactggagtgacacagctgccagaaagcattgtccagctcaagcgactgatgcatcttgttgggcaacggctcatcatgccagacgggtttggtagcatggaatcccttgaggagttaggtactatcgactgctgcaagtgccccgtcagttttggggaagacctagcacttctgagcaggctgagggtgctccgagtggctttcatcggggtcgaaacaagtgacatggaaaccagaaggaaatctttgatgtcatccctctgcaaactcggaggagacaaccttcggcgtgtcactattatcgacctcgctggcggtggagattgctttgtggagtcgtggcaccctcctcctcgtctcctccagaagttcatccatatcagtcagcaacagcacttctccaggtttccagaatggatcagttcctgcctatgtgatctcacccacctggatataaaggccgaaaagatggaaagggagcatctaagtgttcttgaacacctgcccgccatccgttgcctataccttttcgtgaagcgagtctccgaagacgggctcgccatcagccacggcgcgttccgatgtctacggcgtctcgagttctgcaacgtagatggacctggtttgatgtttgcaggaggcgttccaatgttggaatggctgaggctcgggttcgacgcggatagagcgcaatcgacatacggcggtctggaggttggcatccagcgcctctcgtctctcaaacatgtcgtgctcattgtatggatggtttctgaaggcggtgatgatccagcggagcaagccgtctggtctgccatcaatggccaagtagagatgctccccaactctccgacggttgatatccggtttcgtagacggagtcagctgcaggcaagctcagaataa

SEQ ID NO: 8 - DNA, 3124 bp, Zea mays
  source 1..3124: mol_type = genomic DNA
maysatggaaggagctggggaggggagcgccttgacggggatgatgggtcctgcgctcgacaagctcgctagcctcgttgacaagtacaccgagctcagaaacgcgaggaagaagatggagcagctgaggaaggagctgattgccatcaacctcgcgcttgagaagcacgcggccatggagaacccagacgcgcaggcgaaggcgtgggcggcggagatgcgcgagctggcctacgacatggaggacagcatcgatctcttcacccaccacgtcgaccacgaaccggccgacaccgccaccaccggcgtcaagaggttcttcctccggatcatccggaagcttaagaaactccactaccgccacaggtttgctcaggagatcaaacaactccacgaccttgccaacgaatcgtaccggcgtaggaagaggtacaggattgaggagggcggttcaagcctcccgcacgcggagatcgatcctcggttagaggcgctctacgtggaggtggagaaactcgtgggcatccagggcccaagccaggagatcattggacagctcgtcggcgagaacgcagcggagcggcggagggttgtcgccgttgttggatctggaggttcaggcaagaccacacttgccaaacaggtgtacgagaaaatcaggtgccaattctcttgtgcagcctttgtgtccgtgtcgcaaaagcccaacatgaatagcctcctgtgggagttgttatctcaaatcgggaaccatggtggagatttaggaatgatggcagtaggatattgcagtgacaaacaactgatcgacagactaagatcacatcttgaaaagcagaggttagtagtttaccttttcattccggttagcttaattcggtacaccaactagagatttgtgatttgctattaattacaccaaatttctcttacacaacaataactggtttagcatgatggcaatccaaagtcaaaactatcttctactactgtctactagtgtatgccatactcatatagatattttcttttcataaactctcgtagcatttttacatgcattcatattcctattgcctttatacagaactgatttttcaactgcttcacaatctgctcttaggtatctcgttgtgatagatgatgtttggacaaactcagcgtgggagaccatacaatgtgcgctccctaaaaatgcccatgcaagtaaaataattctgacaacacgaatcaacagtgtaggccagttctcctgcactccagatgagggttttatctatcagatgaagcctctttgcagaaacgattctgaaaatctgtttctgaaaaggacactatgtgataaagataagtttcctgctcagctggaggggattaaaaacgagataatcgagaaatgcgatggtttgccactggctattgttactctagctagcatgttagctactaaacagagaacaagggaagaatgggagagggcacttgattcaatccattctacgcacaagaaagatagtagcctggaagtgatggacaagatactgtctctgagttacagggatctacctcacaacatgagaaattgcttgctgtatatcagtacatttccagaggaccacacgatttacaaagatgctctagtatggagatggatggctgaagggtttatcgctgaaacacaaggctttactttggagcaggttgccgagggctacttctacgagtttgtgaacaggagtttggttcagcccataaccttgcgttcaagatatgaaatgcgtggagaaggaggttgccgagtccatgacattgtactgaacttcctcatctctcgtgcagctgaagagaactttttaactacgctgtatggcgcccagggggttccatcttcagaccgaaggattcgccggctctctgtctgggacagtccagaacacgcactggcagtctctagagcgaccatgaatctgtcccatctccggtcagttagaatatgcaacgttggagactggcccgtgcctgctgttctagacttacctgtccttcgagtgttagatctagagggatgccgtgatctgaggatcgtcgaccctgactgcattctaagcttgtttcatctgaggtacctgggtttccgcagcgcaagtggtgtcgtgctaccggctcaaataggaaatttacaccatctgcagaccatcgatttaagcgggactggagtgacacagctgccagaaagcattgtccagctcaagcgactgatgcatcttgttgggcaacggctcatcatgccagacgggtttggtagcatggaatcccttgaggagttaggtactatcgactgctgcaagtgccccgtcagttttggggaagacctggcacttctgagcaggctgagggtgctccgagtgaccgagtggctttcgtcggggtcgaaacaagtgacatggaaaccagaaggaaatctttgatgtcatccctctgcaaactcggaggagacaaccttcggcgtgtcactattatcgacctcgctggcggtggagattgctttgtggagtcgtggcaccctcctcctcgtctcctccagaagttcatccatatcagtcagcaacagcacttctccaggtttccagaatggatcagttcctgcctatgtgatctcacccacctggatataaaggccgaaaagatggaaagggagcatctaagtgttcttgaacacctgcccgccatccgttatctataccttttcgtgaagcgagtctccgaagacgggctcgtcatcagccacagcgcgttccgatgtctacggcgtctcgagttctgtaacttagatggacctggtttgatgtttgcaggaggcgttccaatgctggaatggctgaggctcgggttcgacgcggatagagcgcaatcgacatacggcggtctggaggttggcatccagcgcctctcgtctctcaaacatgtcgtgctcattgtctgtatggtttctgaaggcggtgatgatccagcggagcaagccgtctggtctgccatcaatggccaagtagagatgctccccaattctccgacggttgatatccggtttcgtagacggagtcagctgcaggcaagctcagaataa

(19) United States
(12) Patent Application Publication
(10) Pub. No.: US 2023/0210080 A1
(43) Pub. Date: Jul. 6, 2023

(54) GENERATING MAIZE PLANTS WITH ENHANCED RESISTANCE TO NORTHERN LEAF BLIGHT

(71) Applicant: PIONEER HI-BRED INTERNATIONAL, INC., JOHNSTON, IA (US)

(72) Inventors: ZHENGLIN HOU, ANKENY, IA (US); APRIL L. LEONARD, DES MOINES, IA (US); BAILIN LI, JOHNSTON, IA (US); GIRMA M. TABOR, JOHNSTON, IA (US); SHAWN THATCHER, URBANDALE, IA (US)

(73) Assignee: PIONEER HI-BRED INTERNATIONAL, INC., JOHNSTON, IA (US)

(21) Appl. No.: 18/067,345

(22) Filed: Dec. 16, 2022

Related U.S. Application Data
(63) Continuation of application No. PCT/US21/31741, filed on May 11, 2021.
Application No. PCT/US21/31741 is a continuation-in-part of application No. 17/930,618, filed on Sep. 8, 2022.
Application No. 17/930,618 is a continuation-in-part of application No. 17/319,319, filed on May 13, 2021, now abandoned.
Application No. 17/319,319 is a continuation-in-part of application No. 16/903,814, filed on Jun. 17, 2020, now Pat. No. 11,653,609.
Application No. 16/903,814 is a continuation of application No. 15/765,566, filed on Apr. 3, 2018, now abandoned.
Application No. 15/765,566 is a continuation-in-part of application No. 16/341,531, filed on Apr. 12, 2019.
(60) Provisional application No. 63/040,122, filed on Jun. 17, 2020.
(60) Provisional application No. 62/242,691, filed on Oct. 16, 2015.
(60) Provisional application No. 63/040,122, filed on Jun. 17, 2020.
(60) Provisional application No. 62/407,867, filed on Oct. 13, 2016.

(51) Int. Cl. A01H 6/46; C12N 15/82; C07K 14/415

(52) U.S. Cl. CPC A01H 6/4684; C12N 15/8282; C07K 14/415

(57) ABSTRACT

Compositions and methods for generating maize plants that exhibit resistance to northern leaf blight are provided herein. Polynucleotides encoding a polypeptide that confers resistance to northern leaf blight, polynucleotide constructs comprising such, and maize plants comprising the polynucleotide constructs are provided.

### FIELD

The present disclosure relates to compositions and methods useful in generating maize plants with enhanced resistance to northern leaf blight.

### REFERENCE TO SEQUENCE LISTING SUBMITTED ELECTRONICALLY

The official copy of the sequence listing is submitted electronically via EFS-Web as an ASCII formatted sequence listing with a file named 8493_SeqList.txt created on Jun. 16, 2020 and having a size 46 kilobytes and is filed concurrently with the specification. The sequence listing contained in this ASCII formatted document is part of the specification and is herein incorporated by reference in its entirety.

### BACKGROUND

Northern leaf blight (NLB), induced by the fungal pathogen Exserohilum turcicum (previously called Helminthosporium turcicum), is a serious foliar wilt disease of maize in many tropical and temperate environments. Symptoms can range from cigar-shaped lesions on the lower leaves to complete destruction of the foliage, thereby reducing the amount of leaf surface area available for photosynthesis. A reduction in photosynthetic capability leads to a lack of carbohydrates needed for grain fill, which impacts grain yield. Mid-altitude regions of the tropics, about 900-1600 m above sea level, have a particularly favorable climate for northern leaf blight, as dew periods are long and temperatures moderate. However, northern leaf blight can also yield losses of 30-50% in temperate environments, such as in the United States, during wet seasons, particularly if the infection is established on the upper leaves of the plant by the silking stage.

The most effective and most preferred method of control for northern leaf blight is the planting of resistant hybrids. Several varieties or races of Exserohilum turcicum are present in nature, leaving growers with two hybrid options: partial resistant hybrids, which offer low-level, broad spectrum protection against multiple races, and race-specific resistant hybrids, which protect against a specific race. Genetic sources of resistance to Exserohilum turcicum have been described, and four Exserohilum turcicum resistance loci have been identified: Ht1, Ht2, Ht3, and Htn1. Gene Ht1 maps to the long arm of chromosome 2 where it is closely linked to umc36 (Coe, E. H. et al. (1988), Corn and Corn Improvement, 3rd edn., pp. 81-258), sgcr506 (Gupta, M. et al. (1989) Maize Genet. Coop. Newsl. 63, 112), umc150B (Bentolila, S. et al. (1991) Theor. Appl. Genet., 82:393-398), and pic18a (Collins et al. (1998) Molecular Plant-Microbe Interactions, 11:968-978), and it is closely flanked by umc22 and umc122 (Li et al. (1998) Hereditas, 129:101-106). Gene Ht2 maps to the long arm of chromosome 8 in the umc48-umc89 interval (Zaitlin et al. (1992) Maize Genet. Coop. Newsl., 66, 69-70), and gene Ht3 maps to chromosome 7 near bn1g1666 (Van Staden, D et al. (2001) Maize Genetics Conference Abstracts 43:P134). The Htnl gene maps to chromosome 8, approximately 10 cM distal to Ht2 and 0.8 cM distal to the RFLP marker umc117 (Simcox and Bennetzen (1993) Maize Genet. Coop. Newl. 67, 118-119; Simcox and Bennetzen (1993) Phytopathology, 83:1326-1330).

The methods of controlling northern leaf blight by reducing fungal inoculum require additional time and resources on the part of the farmer, and in addition, can have detrimental effects on the environment. This makes the planting of resistant hybrids even more attractive to farmers and the general public. Thus, it is desirable to provide compositions and methods for generating maize plants with enhanced resistance to northern leaf blight.

### SUMMARY

Presented herein are compositions and methods for generating maize plants exhibiting resistance to northern leaf blight, whether that resistance is newly conferred or enhanced.

Isolated polynucleotides are presented herein that can be used to generate maize plants that exhibit resistance to northern leaf blight. In some embodiments, an isolated polynucleotide may be selected from the group consisting of: (a) the nucleotide sequence set forth in SEQ ID NO:1, 2, or 6; (b) a nucleotide sequence encoding a polypeptide having an amino acid sequence of at least 90% sequence identity when compared to SEQ ID NO:2. In some embodiments, an isolated polynucleotide may comprise a nucleotide sequence encoding a polypeptide having at least 90% identity to SEQ ID NO: 2.

Polynucleotide constructs comprising the isolated polynucleotides are also provided, wherein an isolated polynucleotide is operably linked to a promoter. A polynucleotide construct may further comprise one or more heterologous nucleic acid sequences that encode a polypeptide selected from the group consisting of: a polypeptide conferring disease resistance, a polypeptide conferring herbicide resistance, a polypeptide conferring insect resistance, a polypeptide involved in carbohydrate metabolism, a polypeptide involved in fatty acid metabolism, a polypeptide involved in amino acid metabolism, a polypeptide involved in plant development, a polypeptide involved in plant growth regulation, a polypeptide involved in yield improvement, a polypeptide involved in drought resistance, a polypeptide involved in cold resistance, a polypeptide involved in heat resistance, and/or a polypeptide involved in salt resistance, wherein the one or more heterologous nucleic acid sequences are operably linked to a promoter. For example, a polypeptide conferring disease resistance compared to a control plant may be a polypeptide that confers resistance to northern leaf blight (NLB), which may further have an amino acid sequence of at least 90% sequence identity when compared to SEQ ID NO: 2.

Maize plant cells comprising the polynucleotide constructs and maize plants comprising the maize plant cells are also provided.

Methods for generating maize plants that exhibit resistance to northern leaf blight are provided herein, in which a polynucleotide construct comprising an isolated polynucleotide provided herein, wherein said isolated polynucleotide is operably linked to at least one regulatory sequence, is expressed in a regenerable maize plant cell, and a maize plant that exhibits resistance to northern leaf blight is generated from the maize plant cell. The maize plant generated by the method comprises in its genome the polynucleotide construct. The regulatory sequence may be a promoter and/or a terminator and may be native to maize. In some aspects, the regulatory sequence is native to the Ht1 gene. In still other aspects, the polynucleotide construct comprises one or more additional heterologous nucleic acid sequences that encode a polypeptide selected from the group consisting of: a polypeptide conferring disease resistance, a polypeptide conferring herbicide resistance, a polypeptide conferring insect resistance, a polypeptide involved in carbohydrate metabolism, a polypeptide involved in fatty acid metabolism, a polypeptide involved in amino acid metabolism, a polypeptide involved in plant development, a polypeptide involved in plant growth regulation, a polypeptide involved in yield improvement, a polypeptide involved in drought resistance, a polypeptide involved in cold resistance, a polypeptide involved in heat resistance, and/or a polypeptide involved in salt resistance, wherein each heterologous nucleic acid sequence is operably linked to a promoter. The polypeptide may be one that confers resistance to northern leaf blight (NLB), such as for example, a polypeptide having an amino acid sequence of at least 90% sequence identity when compared to SEQ ID NO: 2. A progeny plant comprising the polynucleotide construct may also be generated by crossing the maize plant generated by the method to a second maize plant that does not comprise in its genome the polynucleotide construct. cl DESCRIPTION OF THE SEQUENCE LISTING SEQ ID NO:1 is the nucleotide sequence of the PH4GP Ht1 Genomic Sequence with Native Promoter and Terminator.

SEQ ID NO:2 is the nucleotide sequence of the PH4GP Ht1 Longer Model CDS Sequence.

SEQ ID NO:3 is the amino acid sequence of the Translation of PH4GP Ht1 Longer Model CDS Sequence.

SEQ ID NO:4 is the amino acid sequence of the Translation of PH4GP Ht1 Shorter Model CDS Sequence.

SEQ ID NO:5 is the nucleotide sequence of the PH4GP Ht1 Shorter Model CDS Sequence. SEQ ID NO:6 is the amino acid sequence of the Translation of PH4GP Ht1 Shorter Model CDS Sequence.

SEQ ID NO:7 is the nucleotide sequence of the PH4GP Ht1 Genomic Sequence from ATG to Stop.

SEQ ID NO:8 is the nucleotide sequence of the B73 Ht1 Genomic Sequence from ATG to Stop.

### DETAILED DESCRIPTION

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof, and so forth. All technical and scientific terms used herein have the same meaning as commonly understood to one of ordinary skill in the art to which this disclosure belongs unless clearly indicated otherwise.

### I. Compositions

### A. Ht1 Polynucleotides and Polypeptides

Mapping of a QTL associated with northern leaf blight resistance on chromosome 2, using a population derived from a cross between northern leaf blight resistant line PH4GP and northern leaf blight susceptible line PH5W4, was described in US2010095395. Presented herein is the cloning of the Ht1 gene in maize and identification of a putative CC-NB-LRR (coiled-coil, nucleotide-binding, leucine-rich repeat) gene as the causal gene. Ht1 genomic and cDNA sequence from PH4GP, the resistant source described in US2010095395, is represented by SEQ ID NOs: 1 and 2, respectively, while the amino acid sequences of the encoded polypeptide is represented by SEQ ID NO: 3.

The Zea mays CC-NB-LRR (coiled-coil, nucleotide-binding, leucine-rich repeat; also referred to as Ht1) gene is a member of a large and complex family of disease resistance genes. The mechanism of NB-LRR protein activation and subsequent signaling in effector triggered immunity is not well understood (Eitas and Dangl. 2010. Curr Opin Plant Biol 13 (4):472-477).

Thus, presented herein are polynucleotides that can be used to generate maize plants with resistance to northern leaf blight. The polynucleotide may be (a) the nucleotide sequence set forth in SEQ ID NO: 1, SEQ ID NO: 2, or SEQ ID NO: 6; (b) a nucleotide sequence encoding a CC-NB-LRR polypeptide having an amino acid sequence of at least 90%, 91%, 92%, 93%, 94%, 95%, 96%, 97%, 98%, or 99% sequence identity when compared to SEQ ID NO: 3; or (c) a nucleotide sequence encoding a CC-NB-LRR polypeptide having an amino acid sequence of at least 90%, 91%, 92%, 93%, 94%, 95%, 96%, 97%, 98%, or 99% sequence identity when compared to SEQ ID NO: 1, SEQ ID NO: 2, or SEQ ID NO: 6; wherein said polypeptide comprises an amino acid sequence having at least 95% sequence identity to the nucleotide sequence set forth in SEQ ID NO: 2.

The use of the term “polynucleotide” is not intended to limit a polynucleotide of the disclosure to a polynucleotide comprising DNA. Polynucleotides may comprise ribonucleotides and combinations of ribonucleotides and deoxyribonucleotides. Such deoxyribonucleotides and ribonucleotides include both naturally occurring molecules and synthetic analogues. The polynucleotides of the disclosure also encompass all forms of sequences including, but not limited to, single-stranded forms, double-stranded forms, hairpins, stem-and-loop structures, and the like.

As used herein, an “isolated” or “purified” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For purposes of this disclosure, “isolated” or “recombinant” when used to refer to nucleic acid molecules excludes isolated unmodified chromosomes. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein. When the polypeptide of the disclosure or a biologically active portion thereof is recombinantly produced, optimally culture medium represents less than about 30%, 20%, 10%, 5%, or 1% (by dry weight) of chemical precursors or non-protein-of-interest chemicals. As used herein, a “recombinant” polynucleotide comprises a combination of two or more chemically linked nucleic acid segments which are not found directly joined in nature. By “directly joined” is intended the two nucleic acid segments are immediately adjacent and joined to one another by a chemical linkage. In specific embodiments, the recombinant polynucleotide comprises a polynucleotide of interest such that an additional chemically linked nucleic acid segment is located either 5′, 3′ or internal to the polynucleotide of interest. Alternatively, the chemically-linked nucleic acid segment of the recombinant polynucleotide can be formed by the deletion of a sequence. The additional chemically linked nucleic acid segment or the sequence deleted to join the linked nucleic acid segments can be of any length, including for example, 1, 2, 3, 4, 5, 6, 7, 8, 9, 10, 15, 20 or greater nucleotides. Various methods for making such recombinant polynucleotides are disclosed herein, including, for example, by chemical synthesis or by the manipulation of isolated segments of polynucleotides by genetic engineering techniques. In specific embodiments, the recombinant polynucleotide can comprise a recombinant DNA sequence or a recombinant RNA sequence. A “recombinant polypeptide” comprises a combination of two or more chemically linked amino acid segments which are not found directly joined in nature. In specific embodiments, the recombinant polypeptide comprises an additional chemically linked amino acid segment that is located either at the N-terminal, C-terminal or internal to the recombinant polypeptide. Alternatively, the chemically-linked amino acid segment of the recombinant polypeptide can be formed by deletion of at least one amino acid. The additional chemically linked amino acid segment or the deleted chemically linked amino acid segment can be of any length, including for example, 1, 2, 3, 4, 5, 6, 7, 8, 9, 10, 15, 20 or amino acids.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100). “Sufficiently identical” is used herein to refer to an amino acid sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity. In some embodiments the sequence identity is against the full length sequence of a polypeptide. The term “about” when used herein in context with percent sequence identity means +/−1.0%.

“Fragments” or “biologically active portions” include polypeptide or polynucleotide fragments comprising sequences sufficiently identical to an Ht1 polypeptide or polynucleotide, respectively, and that exhibit disease resistance when expressed in a plant.

Methods for such manipulations are generally known in the art. For example, amino acid sequence variants of a polypeptide can be prepared by mutations in the DNA. This may also be accomplished by one of several forms of mutagenesis, such as for example site-specific double strand break technology, and/or in directed evolution. In some aspects, the changes encoded in the amino acid sequence will not substantially affect the function of the protein. Such variants will possess the desired activity. However, it is understood that the ability of a Ht1 polypeptide to confer disease resistance may be improved by the use of such techniques upon the compositions of this disclosure.

### B. Polynucleotide Constructs

The Ht1 polynucleotides disclosed herein can be provided in expression cassettes (such as, for example, in the form of polynucleotide constructs) for expression in the plant of interest or any organism of interest. The cassette can include 5′ and 3′ regulatory sequences operably linked to an Ht1 polynucleotide. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the Ht1 polynucleotide to be under the transcriptional regulation of the regulatory regions. The expression cassette may additionally contain selectable marker genes. An expression cassette may be optimized for expression in an organism, such as a maize plant.

“Complement” is used herein to refer to a nucleic acid sequence that is sufficiently complementary to a given nucleic acid sequence such that it can hybridize to the given nucleic acid sequence to thereby form a stable duplex. “Polynucleotide sequence variants” is used herein to refer to a nucleic acid sequence that except for the degeneracy of the genetic code encodes the same polypeptide.

The embodiments also encompass nucleic acid molecules encoding Ht1 polypeptide variants. “Variants” of the Ht1 polypeptide encoding nucleic acid sequences include those sequences that encode the Ht1 polypeptides identified by the methods disclosed herein, but that differ conservatively because of the degeneracy of the genetic code as well as those that are sufficiently identical as discussed above. Naturally occurring allelic variants can be identified with the use of well-known molecular biology techniques, such as polymerase chain reaction (PCR) and hybridization techniques as outlined below. Variant nucleic acid sequences also include synthetically derived nucleic acid sequences that have been generated, for example, by using site-directed mutagenesis but which still encode the Ht1 polypeptides disclosed herein.

The expression cassette can include in the 5′-3′ direction of transcription, a transcriptional and translational initiation region (i.e., a promoter), an Ht1 polynucleotide, and a transcriptional and translational termination region (i.e., termination region) functional in plants. The regulatory regions (i.e., promoters, transcriptional regulatory regions, and translational termination regions) and/or the Ht1 polynucleotide may be native/analogous to the maize plant cell or to each other. Alternatively, the regulatory regions and/or the Ht1 polynucleotide may be heterologous to the maize plant cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, may be native with a maize plant, or may be derived from another source (i.e., foreign or heterologous) with respect to the promoter, the Ht1 polynucleotide, the maize plant, or any combination thereof.

The expression cassettes may additionally contain 5′ leader sequences. Such leader sequences can act to enhance translation. Translation leaders are known in the art and include viral translational leader sequences.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used to express the various Ht1 sequences disclosed herein, including the native promoter of the polynucleotide sequence of interest (such as, for example, the native promoter of the Ht1 gene). The promoters can be selected based on the desired outcome. Such promoters include, for example, constitutive, inducible, tissue-preferred, or other promoters for expression in plants or in any organism of interest. Synthetic promoters can also be used to express Ht1 sequences. Synthetic promoters include for example a combination of one or more heterologous regulatory elements.

A polynucleotide construct may be a recombinant DNA construct. A “recombinant DNA construct” comprises two or more operably linked DNA segments which are not found operably linked in nature. Non-limiting examples of recombinant DNA constructs include a polynucleotide of interest operably linked to heterologous sequences which aid in the expression, autologous replication, and/or genomic insertion of the sequence of interest.

Such heterologous and operably linked sequences include, for example, promoters, termination sequences, enhancers, etc, or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

### C. Maize Plant Cells and Maize Plants

“Maize” refers to a plant of the Zea mays L. ssp. mays and is also known as “corn”. Maize plants, maize plant cells, maize plant parts and seeds, and maize grain having the Ht1 sequences disclosed herein are also provided. In specific embodiments, the plants and/or plant parts have stably incorporated at least one heterologous Ht1 polypeptide disclosed herein. In addition, the plants or organism of interest can comprise multiple Ht1 polynucleotides (i.e., at least 1, 2, 3, 4, 5, 6 or more).

As used herein, the term maize plant includes maize plant cells, maize plant protoplasts, maize plant cell tissue cultures from which maize plants can be regenerated, maize plant calli, maize plant clumps, and maize plant cells that are intact in maize plants or parts of maize plants such as embryos, pollen, ovules, seeds, leaves, flowers, kernels, ears, cobs, husks, stalks, roots, root tips, anthers, and the like. Grain is intended to mean the mature seed produced by commercial growers for purposes other than growing or reproducing the species.

### D. Other Traits of Interest

In some embodiments, the Ht1 polynucleotides disclosed herein may be engineered into a molecular stack. Thus, the various maize plants, maize plant cells and maize seeds disclosed herein can further comprise one or more traits of interest, and in more specific embodiments, the maize plant, maize plant part or maize plant cell is stacked with any combination of polynucleotide sequences of interest in order to create plants with a desired combination of traits.

As used herein, the term “stacked” includes having the multiple traits present in the same plant or organism of interest. In one non-limiting example, “stacked traits” comprise a molecular stack where the sequences are physically adjacent to each other. A trait, as used herein, refers to the phenotype derived from a particular sequence or groups of sequences.

A polynucleotide DNA construct described herein may also comprise one or more heterologous nucleic acid sequences that encode a polypeptide selected from the group consisting of: a polypeptide conferring disease resistance, a polypeptide conferring herbicide resistance, a polypeptide conferring insect resistance, a polypeptide involved in carbohydrate metabolism, a polypeptide involved in fatty acid metabolism, a polypeptide involved in amino acid metabolism, a polypeptide involved in plant development, a polypeptide involved in plant growth regulation, a polypeptide involved in yield improvement, a polypeptide involved in drought resistance, a polypeptide involved in cold resistance, a polypeptide involved in heat resistance, and/or a polypeptide involved in salt resistance, wherein each heterologous nucleic acid sequence is operably linked to a promoter.

A polypeptide conferring disease resistance may be another polypeptide that confers resistance to northern leaf blight (NLB). For example, a polynucleotide DNA construct may comprise a resistant allele of Ht1 and a resistant allele of NLB18 (in WO2011163590). Both PH99N and PH26N are maize lines showing resistance to northern leaf blight that reflect different sources of resistance with respect to the chromosome 8 QTL, as described in application WO2011163 590. A resistant allele of NLB18 may encode a polypeptide having an amino acid sequence of at least 90%, 91%, 92%, 93%, 94%, 95%, 96%, 97%, or 99% sequence identity when compared to an NLB18 sequence disclosed in application

WO2011163590.

### II. Methods of Generating Maize Plants with Northern Leaf Blight Resistance

“Exserohilum turcicum”, previously referred to as Helminthosporium turcicum, is the fungal pathogen that induces northern leaf blight infection. The fungal pathogen is also referred to herein as Exserohilum or Et.

“Disease resistance” (such as, for example, northern leaf blight resistance) is a characteristic of a plant, wherein the plant avoids the disease symptoms that are the outcome of plant-pathogen interactions, such as maize-Exserohilum turcicum interactions. That is, pathogens are prevented from causing plant diseases and the associated disease symptoms, or alternatively, the disease symptoms caused by the pathogen are minimized or lessened.

“Resistance” is a relative term, indicating that the infected plant produces better yield of maize than another, similarly treated, more susceptible plant. That is, the conditions cause a reduced decrease in maize survival and/or yield in a tolerant maize plant, as compared to a susceptible maize plant. One of skill will appreciate that maize plant resistance to northern leaf blight, or the pathogen causing such, can represent a spectrum of more resistant or less resistant phenotypes, and can vary depending on the severity of the infection.

A plant having disease resistance may have 5, 10, 15, 20, 25, 30, 35, 40, 45, 50, 55, 60, 65, 70, 75, 80, 85, 90, 95, or 100% increased resistance to a disease compared to a control plant. In some embodiments, a plant may have 5, 10, 15, 20, 25, 30, 35, 40, 45, 50, 55, 60, 65, 70, 75, 80, 85, 90, 95, or 100% increased plant health in the presence of a disease compared to a control plant

The resistance may be “newly conferred” or “enhanced”. “Newly conferred” or “enhanced” resistance refers to an increased level of resistance against a particular pathogen, a wide spectrum of pathogens, or an infection caused by the pathogen(s). An increased level of resistance against a particular fungal pathogen, such as Et, for example, constitutes “enhanced” or improved fungal resistance. The embodiments of the invention will enhance or improve fungal plant pathogen resistance, such that the resistance of the plant to a fungal pathogen or pathogens will increase, which in turn, will increase resistance to the disease caused by the fungal pathogen. The term “enhance” refers to improve, increase, amplify, multiply, elevate, raise, and the like.

The maize plants generated by the methods described herein may provide durable and broad spectrum resistance to the maize plant and may assist in breeding of northern leaf blight resistant maize plants. For instance, if multiple northern leaf blight resistance genes are stacked into one unit, this reduces the number of specific loci that require trait introgression through backcrossing and minimizes linkage drag from non-elite resistant donors.

Various methods can be used to introduce a sequence of interest into a maize plant cell, maize plant or maize plant part. “Introducing” is intended to mean presenting to the maize plant cell, maize plant, or maize plant part the polynucleotide in such a manner that the sequence gains access to the interior of a cell of the maize plant. The methods of the disclosure do not depend on a particular method for introducing a sequence into an organism or a maize plant or maize plant part, only that the polynucleotide gains access to the interior of at least one cell of the maize plant. Methods for introducing polynucleotides into various organisms, including maize plants, are known in the art, including, but not limited to, stable transformation methods, transient transformation methods, and virus-mediated methods.

“Stable transformation” is intended to mean that the polynucleotide construct introduced into a maize plant integrates into the genome of the maize plant and is capable of being inherited by the progeny thereof. “Transient transformation” is intended to mean that a polynucleotide is introduced into the maize plant and does not integrate into the genome of the maize plant.

Transformation protocols as well as protocols for introducing polynucleotide sequences into plants such as maize may vary. Suitable methods of introducing polynucleotides into maize plant cells include microinjection (Crossway et al. (1986) Biotechniques 4:320 334), electroporation (Riggs et al. (1986) Proc. Natl. Acad. Sci. USA 83:5602 5606, Agrobacterium-mediated transformation (U.S. Pat. Nos. 5,563,055 and 5,981,840), direct gene transfer (Paszkowski et al. (1984) EMBO J. 3:2717 2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923 926); and Lec1 transformation (WO 00/28058). Also see Klein et al. (1988) Proc. Natl. Acad. Sci. USA 85:4305 4309 (maize); Klein et al. (1988) Biotechnology 6:559 563 (maize); U.S. Pat. Nos. 5,240,855; 5,322,783; and, 5,324,646; Klein et al. (1988) Plant Physiol. 91:440 444 (maize); Fromm et al. (1990) Biotechnology 8:833 839 (maize); Hooykaas-Van Slogteren et al. (1984) Nature (London) 311:763-764; U.S. Pat. No. 5,736,369 (cereals); De Wet et al. (1985) in The Experimental Manipulation of Ovule Tissues, ed. Chapman et al. (Longman, New York), pp. 197-209 (pollen); D'Halluin et al. (1992) Plant Cell 4:1495-1505 (electroporation); and Osjoda et al. (1996) Nature Biotechnology 14:745-750 (maize via Agrobacterium tumefaciens); all of which are herein incorporated by reference.

In other embodiments, the Ht1 polynucleotide disclosed herein thereof may be introduced into plants by contacting plants with a virus or viral nucleic acids. Generally, such methods involve incorporating a polynucleotide construct of the disclosure within a DNA or RNA molecule. It is recognized that the Ht1 sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and

WO99/25853, all of which are herein incorporated by reference. Briefly, the polynucleotide disclosed herein can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome. Other methods to target polynucleotides are set forth in WO 2009/114321 (herein incorporated by reference), which describes “custom” meganucleases produced to modify plant genomes, in particular the genome of maize. See, also, Gao et al. (2010) Plant Journal 1:176-187.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide disclosed herein, for example, as part of an expression cassette, stably incorporated into their genome.

Transformed maize plant cells which are derived by plant transformation techniques, including those discussed above, can be cultured to regenerate a whole plant which possesses the transformed genotype (i.e., an Ht1 polynucleotide that encodes a polypeptide that confers resistance to northern leaf blight), and thus the desired phenotype, such as resistance to northern leaf blight, whether that resistance is newly conferred or enhanced. For transformation and regeneration of maize see, Gordon-Kamm et al., The Plant Cell, 2:603-618 (1990). Plant regeneration from cultured protoplasts is described in Evans et al. (1983) Protoplasts Isolation and Culture, Handbook of Plant Cell Culture, pp 124-176, Macmillan Publishing Company, New York; and Binding (1985) Regeneration of Plants, Plant Protoplasts pp 21-73, CRC Press, Boca Raton. Regeneration can also be obtained from plant callus, explants, organs, or parts thereof. Such regeneration techniques are described generally in Klee et al. (1987) Ann Rev of Plant Phys 38:467. See also, e.g., Payne and Gamborg.

One of skill will recognize that after the expression cassette containing the Ht1 gene is stably incorporated in transgenic plants and confirmed to be operable, it can be introduced into other plants by sexual crossing. Any of a number of standard breeding techniques can be used, depending upon the species to be crossed.

In some embodiments, the methods comprise introducing by way of expressing in a regenerable maize plant cell a polynucleotide construct comprising a polynucleotide operably linked to at least one regulatory sequence, wherein the polynucleotide encodes a resistant allele of the Ht1 gene presented herein, and generating a maize plant that has resistance to northern leaf blight from the maize plant cell. The maize plant generated by the method comprises in its genome the polynucleotide construct. The regulatory sequence may be a promoter and/or a terminator and may be native to maize. In some embodiments, the regulatory sequence is native to the Ht1 gene. A progeny plant comprising the polynucleotide construct may also be generated by crossing the maize plant generated by the method to a second maize plant that does not comprise in its genome the polynucleotide construct. In some embodiments, the Ht1 gene is overexpressed (either as a genomic fragment or cDNA) to impart greater resistance than the level of expression in the native state.

In some embodiments, polynucleotide compositions can be introduced into the genome of a plant using genome editing technologies, or previously introduced polynucleotides in the genome of a plant may be edited using genome editing technologies. For example, the identified polynucleotides can be introduced into a desired location in the genome of a plant through the use of double-stranded break technologies such as TALENs, meganucleases, zinc finger nucleases, CRISPR-Cas, and the like. For example, the identified polynucleotides can be introduced into a desired location in a genome using a CRISPR-Cas system, for the purpose of site-specific insertion. The desired location in a plant genome can be any desired target site for insertion, such as a genomic region amenable for breeding or may be a target site located in a genomic window with an existing trait of interest. Existing traits of interest could be either an endogenous trait or a previously introduced trait.

In some embodiments, where a Ht1 allele has been identified in a genome, genome editing technologies may be used to alter or modify the polynucleotide sequence. Site specific modifications that can be introduced into the desired Ht1 allele polynucleotide include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. Such technologies can be used to modify the previously introduced polynucleotide through the insertion, deletion or substitution of nucleotides within the introduced polynucleotide. Alternatively, double-stranded break technologies can be used to add additional nucleotide sequences to the introduced polynucleotide. Additional sequences that may be added include, additional expression elements, such as enhancer and promoter sequences. In another embodiment, genome editing technologies may be used to position additional disease resistant proteins in close proximity to the Ht1 polynucleotide compositions within the genome of a plant, in order to generate molecular stacks disease resistant proteins.

An “altered target site,” “altered target sequence.” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

### EXAMPLES

The following examples are offered to illustrate, but not to limit, the appended claims. It is understood that the examples and embodiments described herein are for illustrative purposes only and that persons skilled in the art will recognize various reagents or parameters that can be altered without departing from the spirit of the invention or the scope of the appended claims.

### Example 1. PH4GP Genomic Fragment Containing the Ht1 Candidate Gene Enhance NLB Resistance

To validate the Ht1 candidate gene, a construct was made with the PH4GP Ht1 genomic fragment (SEQ ID NO: 1), which includes the native promoter, the genomic candidate gene sequence, and the native terminator. This construct was transformed into a NLB susceptible line, and segregating material was screened for NLB resistance with a greenhouse-based NLB assay. Forty-eight transgene-positive plants were NLB resistant, while 9 nulls were susceptible, thus confirming that the genomic fragment of the candidate gene can confer resistance to NLB.

### Example 2. PH4GP CDS of the Ht1 Candidate Gene Confers NLB Resistance

Two gene models for the validate PH4GP genomic fragment were predicted. The coding DNA sequences (CDS) of both models were tested to determine which one would confer NLB resistance. One gene model was 2829 nucleotides (SEQ ID NO: 2), resulting in a protein 944 amino acids, including the stop codon (SEQ ID NO: 3). The other gene model was 2658 nucleotides (SEQ ID NO: 4) and the corresponding protein is 886 amino acids including the stop codon (SEQ ID NO: 5). The structure of these two predicted gene models was the same except that the shorter model was truncated by 171 nucleotides from the 5′ end. Constructs for both gene models were made with the CDS under the control of the maize H2B promoter. The constructs were transformed into a NLB susceptible line, and segregating material was screened for NLB resistance. All plants containing the shorter CDS (SEQ ID NO: 4) and the nulls were susceptible. Nine transgenic plants containing the longer CDS (SEQ ID NO: 2) were resistant to NLB while 9 nulls were susceptible. This confirmed that only the longer CDS (SEQ ID NO: 2) is functional, and the shorter CDS (SEQ ID NO: 4) was not efficacious as tested.

### Example 3. Sequence Variation Between Resistance and Susceptible Alleles is the Causal Variation for NLB Resistance

The Ht1 gene from the resistant line PH4GP is expressed at a much higher much level than the Ht1 allele from the susceptible line B73. To test whether the sequence or expression variation is the causal variation for NLB resistance, transgenic plants expressing the PH4GP or B73 Ht1 allele driven by the same promoter were generated. Specifically, we made constructs in which both the PH4GP Ht1 genomic sequence from the ATG to the stop codon (SEQ ID NO: 6) and the B73 Ht1 genomic sequence from the ATG to the stop codon (SEQ ID NO: 7) were driven by the maize H2B promoter. Both constructs were transformed into a NLB susceptible line, and segregating material was screened for NLB resistance. All plants containing the B73 allele, as well as the nulls, were susceptible to NLB. In contrast, all 8 plants containing the PH4GP allele were resistant to NLB, while the corresponding 9 nulls were susceptible. The expression levels of the PH4GP and B73 Ht1 alleles in the transgene-positive plants were comparable. This indicates that sequence variation between the PH4GP (resistant) and B73 (susceptible) alleles, rather than expression variations, determines the NLB resistance.

What is claimed is:

1. A DNA construct comprising a polynucleotide selected from the group consisting of:
a. a nucleotide sequence having at least 90% sequence identity when compared to set forth in any one of SEQ ID NOs: 1, 2, or 6; or
b. a nucleotide sequence encoding a polypeptide having an amino acid sequence having at least 90% sequence identity when compared to SEQ ID NO: 3;
wherein said polynucleotide is operably linked to a promoter.

2. The DNA construct of claim 1, wherein the DNA construct further comprises one or more additional heterologous nucleic acid sequences that encode a polypeptide selected from the group consisting of: a polypeptide conferring disease resistance, a polypeptide conferring herbicide resistance, a polypeptide conferring insect resistance, a polypeptide involved in carbohydrate metabolism, a polypeptide involved in fatty acid metabolism, a polypeptide involved in amino acid metabolism, a polypeptide involved in plant development, a polypeptide involved in plant growth regulation, a polypeptide involved in yield improvement, a polypeptide involved in drought resistance, a polypeptide involved in cold resistance, a polypeptide involved in heat resistance, and/or a polypeptide involved in salt resistance, wherein each heterologous nucleic acid sequence is operably linked to a promoter.

3. The polynucleotide construct of claim 2, wherein a polypeptide conferring disease resistance is a polypeptide that confers resistance to northern leaf blight (NLB).

4. A maize plant cell comprising the polynucleotide construct of claim 1.

5. A maize plant comprising the maize plant cell of claim 4.

6. A method for producing a maize plant that exhibits resistance to northern leaf blight (NLB) comprising,
a. expressing in a regenerable maize plant cell a heterologous polynucleotide construct comprising a polynucleotide operably linked to at least one regulatory sequence, wherein the polynucleotide is selected from the group consisting of:
i. a nucleotide sequence having at least 90% sequence identity when compared to set forth in any one of SEQ ID NOs: 1, 2, or 6; or
ii. a nucleotide sequence encoding a polypeptide having an amino acid sequence having at least 90% sequence identity when compared to SEQ ID NO: 3; and
b. generating a maize plant that exhibits resistance to northern leaf blight, wherein said maize plant comprises in its genome the polynucleotide construct.

7. The method of claim 6, wherein said at least one regulatory sequence is a promoter.

8. The method of claim 6, wherein said at least one regulatory sequence is a terminator.

9. The method of claim 6, wherein said regulatory sequence is native to maize.

10. The method of claim 6, wherein said regulatory sequence is native to the Ht1 gene.

11. The method of claim 6, wherein said polynucleotide construct comprises one or more additional heterologous nucleic acid sequences that encode a polypeptide selected from the group consisting of: a polypeptide conferring disease resistance, a polypeptide conferring herbicide resistance, a polypeptide conferring insect resistance, a polypeptide involved in carbohydrate metabolism, a polypeptide involved in fatty acid metabolism, a polypeptide involved in amino acid metabolism, a polypeptide involved in plant development, a polypeptide involved in plant growth regulation, a polypeptide involved in yield improvement, a polypeptide involved in drought resistance, a polypeptide involved in cold resistance, a polypeptide involved in heat resistance, and/or a polypeptide involved in salt resistance, wherein each heterologous nucleic acid sequence is operably linked to a promoter.

12. The method of claim 11, wherein the polypeptide conferring disease resistance is a polypeptide that confers resistance to northern leaf blight (NLB).

13. A method of obtaining a maize plant that exhibits resistance to northern leaf blight (NLB), said method comprising,
a. crossing a maize plant generated by the method of claim 6 with a maize plant that does not comprise in its genome the polynucleotide construct;
b. obtaining a progeny plant that exhibits resistance to northern leaf blight, wherein said progeny plant comprises the polynucleotide construct in its genome.
